(19) United States
(12) Patent Application Publication
(10) Pub. No.: US 2021/0015837 A1
(43) Pub. Date: Jan. 21, 2021

(54) Method of Manufacturing an antiviral super treatment

(71) Applicant: Vivi R. Stafford, Mission Viejo, CA (US)

(72) Inventor: Vivi R. Stafford, Mission Viejo, CA (US)

(21) Appl. No.: 16/927,914

(22) Filed: Jul. 13, 2020

Related U.S. Application Data
(63) Continuation-in-part of application No. 16/842,787, filed on Apr. 8, 2020.

(51) Int. Cl. A61K 31/7052; A61K 31/65; A61P 31/14; A01N 37/18; A01N 43/72

(52) U.S. Cl. CPC A61K 31/7052; A61K 31/65; A01N 43/72; A01N 37/18; A61P 31/14

(57) ABSTRACT

Method of Manufacturing an antiviral super pill or syrup for the treatment of viral infection such as SARS-CoV-2 (Covid 19). A cyclin antibiotic as well as a macrolide antibiotic are combined within the same compounded pill or capsule for the treatment of viruses. Doxycycline, a cyclin, binds to the 30S ribosomal subunit, and presumably to the 50S ribosomal subunit, thereby blocking the binding of aminoacyl-tRNA to the mRNA-ribosome complex, Azithromycin, a macrolide, reversibly binds to the 50S ribosomal subunit of the 70S ribosome of sensitive microorganisms, thereby inhibiting the translocation step of protein synthesis, wherein a newly synthesized peptidyl tRNA molecule moves from the acceptor site on the ribosome to the peptidyl (donor) site, and consequently inhibiting RNA-dependent protein synthesis leading to cell growth inhibition and cell death. As a result, this combination can provide immunity from certain viruses, such as Coronavirus 2019 (Covid 19) when given at the same time of a nasopharyngeal exposure to virus particles within numbers greater or less than 1000 virions, as a treatment regimen for a cure or as a prophylaxis for those exposed or were presumably exposed.

Method of Manufacturing an antiviral super pill or syrup for the treatment of viral infection such as SARS-CoV-2 (Covid 19). A cyclin antibiotic as well as a macrolide antibiotic are combined within the same compounded pill or capsule for the treatment of viruses. Doxycycline, a cyclin, binds to the 30S ribosomal subunit, and presumably to the 50S ribosomal subunit, thereby blocking the binding of aminoacyl-tRNA to the mRNA-ribosome complex. Azithrornycin, a macrolide, reversibly binds to the 50S ribosomal subunit of the 70S ribosome of sensitive microorganisms, thereby inhibiting the translocation step of protein synthesis, wherein a newly synthesized peptidyl tRNA molecule moves from the acceptor site on the ribosome to the peptidyl (donor) site, and consequently inhibiting RNA-dependent protein synthesis leading to cell growth inhibition and cell death. As a result, this combination can provide immunity from certain viruses, such as Coronavirus 2019 (Covid 19) when given at the same time of a nasopharyngeal exposure to virus particles within numbers greater or less than 1000 virions, as a treatment regimen for a cure or as a prophylaxis for those exposed or were presumably exposed.

### BRIEF DESCRIPTION OF THE INVENTION

Aminoacyl-tRNA synthetases play a pivotal role in protein synthesis and cell viability. An aminoacyl-tRNA synthetase (aaRS or ARS), also called tRNA-ligase, is an enzyme that attaches the appropriate amino acid onto its tRNA. It does so by catalyzing the esterification of a specific cognate amino acid or its precursor to one of all its compatible cognate tRNAs to form an aminoacyl-tRNA. Other viruses mimic tRNA-like structures in their genomes, and these motifs are aminoacylated by the host synthetase as part of the viral replication cycle.

### DETAILED DESCRIPTION OF THE INVENTION

Viruses, more specifically Covid 19 or the Coronavirus with an initial outbreak in Wuhan China in 2019, the virus mimic's tRNA-like structures from antibiotic influenced aminoacyl-tRNA which has the ability to interrupt the appropriate binding of an amino acid into its RNA in relations to specific viruses. In addition, the Doxycycline has a negative impact on the aminoacylation of the host synthetase; therefore, interrupting the Covid 19 viruses (or other susceptible viruses) the opportunity to reproduce. SARS-CoV-2 virus the aminoacyl tRNA then co-opts proteins, reassigning them to the task of making millions of copies of itself. However, viral amplification cannot be successful as the Doxycycline inhibits the attachment of the appropriate amino acid necessary for the virus's replication. Therefore, the virus does not have the opportunity of being copied causing cell injury and death. There are zero risks for outbreaks through the use of Doxycycline. Azithromycin binds to the 50S subunit of the bacterial ribosome, thus inhibiting translation of mRNA. Therefore, SARS-CoV-2 virus cannot survive coadministration of Doxycyline and Azithromycin as the functioning of tRNA is interrupted.

### SPECIFICATION

Combination of a cyclin and a macrolide for full coverage for diminishing viral activity in relations to inhibiting the hosts genetic material such as mRNA and tRNA from being utilized by viruses or the SARS-Cov-2 virus. Ultimately, virus's such as Covid 19 will not survive in vivo when Doxycycline and Azithromycin are consumed by virus exposed and infected individuals. A superbill is used which encompasses a cyclin such as Doxycycline taken twice daily compounded with Azithromycin where supplemental zinc may or may not be compounded within to rid susceptible viral infections such as Coronavirus 19.

1. Compounding together for a cyclin such as Doxycycline 25 to 100 mg twice a day for the treatment of viral infections including Coronavirus or Covid 19 in combination with a Macrolid such as Azithromycin 250 mg daily either in a tablet, capsule, liquid suspension or packet.

2. Vaccination of humans infected with a virus including SARS-CoV-19 through the intentional exposure to a patient of virions simultaneously or followed by the administration of a cyclin such as Doxycyline and a macrolid such as Azithromycin.

3. Compounding of a cyclin such as Doxycycline and a macrolid such as Azithromycins for biofilm treatment such as an approach in decreasing the incidence of drug resistant strands to germs.
